(19) United States
(12) Patent Application Publication
(10) Pub. No.: US 2015/0031737 A1
(43) Pub. Date: Jan. 29, 2015

(54) STABILIZED CONTROLLED-RELEASE PHARMACEUTICAL COMPOSITION COMPRISING GLICLAZIDE

(71) Applicant: RANBAXY LABORATORIES LIMITED, New Delhi, Delhi (IN)

(72) Inventors: Ravindra Agarwal, Udaipur (IN); Ajay Kumar Dobhal, Pauri Garhwal (IN); Ravi Kochhar, Gurgaon (IN)

(21) Appl. No.: 14/380,237

(22) PCT Filed: Feb. 23, 2013
(86) PCT No.: PCT/IB2013/051467

§ 371 Date: Aug. 21, 2014

(30) Foreign Application Priority Data
Feb. 24, 2012 (IN) 538/DEL/2012

(51) Int. Cl. A61K 9/20; B29C 43/00; A61K 31/403

(52) U.S. Cl. CPC A61K 9/2013; A61K 9/2054; A61K 31/403; A61K 9/2095; B29C 43/003; B29K 2001/08
USPC 514/412; 264/118

(57) ABSTRACT

The present invention provides a stabilized controlled-release pharmaceutical composition comprising gliclazide and sodium citrate as stabilizing agent; and process for the preparation of said pharmaceutical composition.

### FIELD OF THE INVENTION

The present invention relates to a stabilized controlled-release pharmaceutical composition comprising gliclazide or its pharmaceutically acceptable salts and sodium citrate as a stabilizing agent.

### BACKGROUND OF THE INVENTION

Gliclazide is a weakly acidic drug with a pKa of about 5.8, having a hydrophobic nature. It belongs to Class II of the biopharmaceutical classification in which dissolution is the rate-controlling step in drug absorption.

Diamicron® MR is marketed as a modified-release tablet of gliclazide. It is formulated with using inactive ingredients including calcium hydrogen phosphate dihydrate, maltodextrin, hypromellose, lactose monohydrate, magnesium stearate, and anhydrous colloidal silicon dioxide.

U.S. Pat. No. 6,733,782 discloses a matrix tablet for the prolonged release of gliclazide, which provides continuous and consistent release of the active ingredient after oral administration, wherein the release is insensitive to variations in the pH of the dissolution medium.

During stability studies, it has been found that Diamicron® MR generates a high amount of Impurity A, (i.e., p-toluenesulphonamide) originating from the decomposition of gliclazide in the pharmaceutical composition. This degradation may be attributed to the presence of certain excipients like calcium hydrogen phosphate dihydrate and/or colloidal silicon dioxide.

PCT Publication No. WO 2008/062470 is directed to a stabilized controlled-release dosage form of gliclazide, wherein the dosage form is without a saccharide component and is optionally free from binder. However, there is a need for an alternate formulation which further provides improved stability over the solid oral formulations of gliclazide available in the prior art.

### SUMMARY OF THE INVENTION

The present invention provides a stabilized controlled-release pharmaceutical composition comprising gliclazide or its pharmaceutically acceptable salts and sodium citrate as a stabilizing agent; wherein, the term “stabilized” refers to a pharmaceutical composition which, when subjected to conditions of 40° C./75% RH for a period of three months, results in total content of Impurity A originating from the decomposition of gliclazide in an amount less than 0.25% w/w.

According to one aspect of the present invention, there is provided a stabilized controlled-release pharmaceutical composition comprising:

a) gliclazide;

b) sodium citrate as a stabilizing agent; and

c) release-controlling polymer(s).

According to another aspect of the present invention, there is provided a stabilized controlled-release pharmaceutical composition comprising:

a) gliclazide;

b) sodium citrate as a stabilizing agent;

c) release-controlling polymer(s); and

d) other pharmaceutically acceptable excipients; wherein the pharmaceutically acceptable excipients are free from colloidal silicon dioxide.

According to another aspect, there is provided a stabilized controlled-release pharmaceutical composition comprising:

a) gliclazide;

b) sodium citrate as a stabilizing agent;

c) two or more release-controlling polymers having different viscosities; and

d) other pharmaceutically acceptable excipients; wherein the pharmaceutically acceptable excipients are free from colloidal silicon dioxide.

A particular aspect of the present invention provides a stabilized controlled-release pharmaceutical composition comprising:

a) gliclazide in an amount ranging from 30 mg to 80 mg;

b) sodium citrate as a stabilizing agent in a concentration from 0.5% w/w to 3.0% w/w;

c) two different viscosity grades of hydroxypropyl methylcellulose, a grade having a viscosity ranging from 100 cps to 750 cps, and a second grade having a viscosity ranging from 1000 cps to 5000 cps; and

d) other pharmaceutically acceptable excipients; wherein the pharmaceutically acceptable excipients are free from colloidal silicon dioxide.

According to yet another aspect of the present invention, there is provided a process for the preparation of a stabilized controlled-release pharmaceutical composition of gliclazide, the process steps comprising of:

a) blending gliclazide with other pharmaceutically acceptable excipients;

b) mixing sodium citrate with the blend of step a);

c) optionally granulating the blend of step b);

d) lubricating the blend of step b) or granules of step c), and compressing into suitable size tablets or filling into capsules; wherein the pharmaceutically acceptable excipients are free from colloidal silicon dioxide.

A further aspect of the present invention provides a process for the preparation of a stabilized controlled-release pharmaceutical composition of gliclazide, the process steps comprising of:

a) sifting gliclazide and other pharmaceutically acceptable excipients, and dry mixing to achieve a uniform blend;

b) dissolving sodium citrate, and optionally a binder, in purified water to form a solution or dispersion;

c) granulating the blend of step a) using the solution or dispersion of step b);

d) sifting the release-controlling polymer using an appropriate sieve and mixing with the dried granules of step c);

e) lubricating the blend of step d); and

f) compressing the lubricated granules of step e) into tablets of suitable size, or optionally filling into capsules; wherein the pharmaceutically acceptable excipients are free from colloidal silicon dioxide.

### DETAILED DESCRIPTION OF THE INVENTION

The term “stabilized”, as used herein, refers to a pharmaceutical composition which, when subjected to conditions of 40° C./75% RH for a period of three months, results in total content of Impurity A originating from the decomposition of gliclazide in an amount less than 0.25% w/w.

“Impurity A”, as used herein, refers to p-toluenesulphonamide originating from the decomposition of gliclazide in the pharmaceutical composition. The relative retention time (RRT) of Impurity A with respect to the gliclazide peak is at 0.18, as per the analytical method used herein, and described later.

The term “controlled-release”, as used herein, refers to the release of an active ingredient from a pharmaceutical composition in which the active ingredient is released according to a desired profile over an extended period of time, and is taken to encompass sustained-release, modified-release, prolonged-release, delayed-release, and the like.

The term “pharmaceutical composition”, as used in this specification, refers to physically discrete units to be administered in single or multiple dosages, each unit containing a predetermined quantity of active material in association with the required pharmaceutically acceptable excipients. The pharmaceutical composition used herein may be selected from tablets, capsules, sachets, pellets, beads, microspheres, microcapsules, or granules.

The term “gliclazide”, as used herein, includes gliclazide free base and pharmaceutically acceptable salts, solvates, hydrates, or mixtures thereof. The pharmaceutical composition of the present invention comprises gliclazide in a range of about 1 mg to about 300 mg. In particular, the compositions may contain about 10 mg to about 200 mg of gliclazide. Particularly, the pharmaceutical composition may contain from 30 mg to 80 mg of gliclazide.

The stabilizing agent added to the composition is sodium citrate. In particular, sodium citrate in a concentration of 0.2% w/w to 5.0% w/w of the composition provides stability to the pharmaceutical composition. Particularly, the pharmaceutical composition contains 0.5% w/w to 3.0% w/w of sodium citrate. The effect of sodium citrate in stabilizing the composition is shown in Table 1, which provides results of stability studies. The compressed tablets, prior to being packed in blister strips, were subjected to stability testing at 40° C./75% RH for one month. The samples were analyzed initially, at an interval of fifteen days, and then at one month. The tablets containing sodium citrate (Example 1) were found to be more stable as compared to the marketed formulation as well as to the formulation without sodium citrate (Example 3).

TABLE 1
Percentage of Impurity A after exposing tablets in open
condition (without blister pack) at 40° C./75% RH

| | Impurity A (% w/w) | | |
|---|---|---|---|
| Formulation | Initial | 15 Days | 30 days |
| Marketed Tablet—Diamicron ® | 0.06 | 0.19 | 0.28 |
| MR 60 mg | | | |
| Example 1 (containing 1.0% w/w | 0.02 | 0.07 | 0.11 |
| sodium citrate) | | | |
| Example 3 (without sodium citrate) | 0.02 | 0.13 | 0.20 |

The controlled-release pharmaceutical composition of the present invention further comprises release-controlling polymer(s), and optionally, other pharmaceutically acceptable excipients.

The release-controlling polymer(s), as used herein, is selected from methylcellulose, ethylcellulose, hydroxyethylcellulose, propylcellulose, hydroxypropylcellulose, hydroxypropyl methylcellulose (hypromellose), carboxymethylcellulose, polymethyl methacrylate, polyethyl methacrylate, polybutyl methacrylate, polyisobutyl methacrylate, polyhexyl methacrylate, polyisodecyl methacrylate, polylauryl methacrylate, polyphenyl methacrylate, polymethyl acrylate, polyisopropyl acrylate, polyisobutyl acrylate, polyoctadecyl acrylate, polyethylene, polypropylene, polyethylene oxide, polyethylene terephthalate, polyvinyl isobutyl ether, polyvinyl acetate, polyvinyl chloride, polyurethane, or mixtures thereof.

In particular, the release-controlling polymer is selected from hydroxyethylcellulose, hydroxypropylcellulose, and hydroxypropyl methylcellulose (hypromellose, HPMC). The release-controlling polymer may be hydroxypropyl methylcellulose. The amount of polymer may vary from 10% w/w to 40% w/w of the total weight of the composition. A particularly used concentration may be from 25% w/w to 35% w/w.

The release-controlling polymer may be a combination of two or more polymers having different viscosities. In particular, two or more different viscosity grades of hydroxypropyl methylcellulose may be used; a grade having a viscosity ranging from 100 cps to 750 cps, and a second grade having a viscosity ranging from 1000 cps to 5000 cps.

Further, the polymer may be added intragranularly and/or extragranularly.

The other pharmaceutically acceptable excipients are selected from diluents, binders, lubricants, or combinations thereof. The excipients used in the formulation may be free from colloidal silicon dioxide.

Binders may be selected from pregelatinized starch, copovidone, shellac, zein, gelatin, polymethacrylates, synthetic resins, Eudragit®, and cellulose polymers.

Diluents may be selected from lactose, calcium hydrogen phosphate dihydrate, calcium hydrogen phosphate anhydrous, tribasic calcium phosphate, calcium carbonate, kaolin, magnesium carbonate, and magnesium oxide.

Lubricants may be selected from stearic acid, polyethylene glycol, magnesium stearate, calcium stearate, zinc stearate, talc, hydrogenated castor oil, and sodium stearyl fumarate. In particular, the lubricant used herein is magnesium stearate.

The pharmaceutical compositions, as described herein, may be prepared by any of the known processes such as direct compression, dry granulation, or wet granulation.

According to one of the embodiments, there is provided a method of preparation of the pharmaceutical composition, the process comprising the steps of:

a) blending gliclazide with other pharmaceutically acceptable excipients;

b) mixing sodium citrate with the blend of step a);

c) optionally granulating the blend of step b); and

d) lubricating the blend of step b) or granules of step c), and compressing into suitable size tablets or filling into capsules.

In particular, another embodiment provides a process for the preparation of a stabilized controlled-release pharmaceutical composition of gliclazide, the process comprising the steps of:

a) sifting gliclazide, diluent, a part of the binder, and release-controlling polymer, and dry mixing to achieve a uniform blend;

b) dissolving sodium citrate and the remaining part of the binder in purified water to form a homogenous slurry;

c) granulating the dry powder blend of step a) using the slurry of step b);

d) sifting the controlled-release polymer using an appropriate sieve and mixing with the dried granules of step c);

e) lubricating the blend of step d);

f) compressing the lubricated granules of step e) into tablets, or optionally filling into capsules.

The tablets prepared using any of the processes described herein may be further coated with a film-coating. Suitable coating compositions comprise film-forming polymer(s), plasticizer(s), opacifier(s), and film smoothener(s). Additionally, pharmaceutically acceptable colors and lakes may be used.

In particular, film-forming polymers like various grades of hydroxypropyl methylcellulose; plasticizer such as a glycol, e.g., propylene glycol, or polyethylene glycol; opacifier such as titanium dioxide; and film smoothener such as talc may be used. Suitable coating solvents are water or organic solvents selected from ethanol, isopropanol, acetone, and halogenated hydrocarbons, or mixtures thereof.

The invention is further illustrated by the following non-limiting examples.

### EXAMPLES

### Example 1

| Ingredients | Quantity (mg/tablet) |
|---|---|
| Gliclazide | 60.00 |
| Pregelatinized starch | 16.00 |
| Lactose monohydrate | 144.20 |
| Sodium citrate | 3.20 |
| Purified water | q.s. |
| Hypromellose | 95.00 |
| Magnesium stearate | 1.60 |
| Total Weight | 320.00 |

### Manufacturing Process:

1. Gliclazide, a part of pregelatinized starch, and lactose monohydrate were sifted and mixed to obtain a uniform blend;
2. sodium citrate was dissolved in purified water followed by the remaining part of the pregelatinized starch to form a homogeneous slurry;
3. the blend of step 1 was transferred into a rapid mixer granulator, and granulated using the slurry of step 2;
4. hypromellose was sifted and mixed with the granules of step 3;
5. the blend of step 4 was lubricated using magnesium stearate; and
6. the lubricated granules of step 5 were compressed into tablets of suitable size using appropriate tooling.

### Example 2

| Ingredients | Quantity (mg/tablet) |
|---|---|
| Gliclazide | 60.00 |
| Pregelatinized starch | 16.00 |
| Lactose monohydrate | 159.20 |
| Sodium citrate | 3.20 |
| Purified water | q.s. |
| Hypromellose K100 LV | 70.00 |
| CR premium | |
| Hypromellose K4M CR | 10.00 |
| Magnesium stearate | 1.60 |
| Total Weight | 320.00 |

### Manufacturing Process:

1. Gliclazide, a part of pregelatinized starch, and lactose monohydrate were sifted and mixed to obtain a uniform blend;
2. sodium citrate was dissolved in purified water followed by the remaining part of the pregelatinized starch to form a homogeneous slurry;
3. the blend of step 1 was transferred into a rapid mixer granulator and granulated using the slurry of step 2;
4. hypromellose K100 LV CR and Hypromellose K4M CR were sifted and mixed with the granules of step 3;
5. the blend of step 4 was lubricated using magnesium stearate; and
6. the lubricated granules of step 5 were compressed into tablets of suitable size using appropriate tooling.

### Example 3: (Control Example)

| Ingredients | Quantity (mg/tablet) |
|---|---|
| Gliclazide | 60.00 |
| Hypromellose E5 Premium LV | 6.40 |
| Lactose monohydrate | 157.00 |
| Purified water | q.s. |
| Hypromellose (Benecel ™ K750 | 95.00 |
| PH PRM) | |
| Magnesium stearate | 1.60 |
| Total Weight | 320.00 |

### Manufacturing Process:

1. Gliclazide, hypromellose E5 Premium LV, and lactose monohydrate were sifted and mixed to obtain a uniform blend;
2. the blend of step 1 was transferred into a rapid mixer granulator and granulated using purified water;
3. hypromellose (Benecel™ K750 PH PRM) was sifted, and mixed with the granules of step 2;
4. the blend of step 3 was lubricated using magnesium stearate; and
5. the lubricated granules of step 4 were compressed into tablets of suitable size using appropriate tooling.

### Stability Testing

The compressed tablets, after being packed in cold-form blisters, were subjected to stability testing at 40° C./75% RH for three months. The samples were analyzed initially, and later at intervals of one month and three months for determining the amount of impurity generated and the extent of degradation. The method for determining related substances employed high performance liquid chromatography (HPLC) using Lichrospher RP-8e 5 μm (250 mm*4 mm) column, and the mobile phase as buffer (pH 2.5):acetonitrile in a ratio of 65:35. The buffer was prepared by adding 2 mL triethylamine to 1 L water, and adjusting the pH to 2.5 using orthophosphoric acid. The relative retention time (RRT) of Impurity A with respect to the gliclazide peak was observed at 0.18.

The marketed tablets (Diamicron® MR), and control tablets (without sodium citrate, Example 3) were also subjected to similar stability testing protocol, and assessed for impurities. The comparative results of the stability study are shown in Table 1 (for tablets subjected to stability testing in an open condition), and Table 2 (for tablets packed in cold-form blister packs), and subjected to stability study protocol.

It was observed that there was more degradation in open conditions than in blister packs, as the packing further provides protection from the effects of heat and moisture.

As seen from Table 2, the tablets of Example 1 and Example 2 (containing sodium citrate) have the least amount of Impurity A, thereby being more stable.

TABLE 2
Percentage of Impurity A after subjecting tablets packed
in cold-form blister packs to 40° C./75% RH

| | Impurity A (% w/w) | | |
|---|---|---|---|
| Formulation | Initial | 1 month | 3 months |
| Marketed Tablet—Diamicron ® | 0.06 | — | 0.57 |
| MR 60 mg | | | |
| Example 1 | 0.02 | 0.09 | 0.22 |
| Example 2 | 0.01 | 0.05 | 0.15 |
| Example 3 | 0.02 | 0.13 | 0.31 |

### Dissolution Study

Table 3 shows the comparison of dissolution profiles of the formulations of Examples 1, 2, and 3. The dissolution was performed in 900 mL of buffer (pH 7.4) at 100 rpm using the basket (40 mesh) method. The samples were analyzed using HPLC using Lichrospher RP-8e 5 μm (250 mm*4 mm) column, and the mobile phase as buffer (pH 2.5):acetonitrile in a ratio of 60:40. The target dissolution profile at the 6 hour time point was set as 40% to 65% of drug-release under the given dissolution conditions. As seen from the results, there is no difference in the dissolution of the formulation from Examples 1, 2, and 3; thereby suggesting that sodium citrate does not affect dissolution.

TABLE 3
Percentage of drug released in dissolution media
of pH 7.4 phosphate buffer

| Time point (hours) | Example 1 | Example 2 | Example 3 |
|---|---|---|---|
| 1 | 9 | 9 | 9 |
| 2 | 19 | 20 | 19 |
| 4 | 39 | 39 | 40 |
| 6 | 55 | 52 | 60 |
| 12 | 84 | 78 | 97 |

We claim:

1. A stabilized controlled-release pharmaceutical composition comprising:
a) gliclazide;
b) sodium citrate as a stabilizing agent; and
c) at least one release-controlling polymer.

2. The stabilized controlled-release pharmaceutical composition of claim 1, wherein sodium citrate is present in a concentration from 0.5% w/w to 3.0% w/w.

3. The stabilized controlled-release pharmaceutical composition of claim 1, wherein the at least one release-controlling polymer is selected from methylcellulose, ethylcellulose, hydroxyethylcellulose, propylcellulose, hydroxypropylcellulose, hydroxypropyl methylcellulose, carboxymethylcellulose, polymethyl methacrylate, polyethyl methacrylate, polybutyl methacrylate, polyisobutyl methacrylate, polyhexyl methacrylate, polyisodecyl methacrylate, polylauryl methacrylate, polyphenyl methacrylate, polymethyl acrylate, polyisopropyl acrylate, polyisobutyl acrylate, polyoctadecyl acrylate, polyethylene, polypropylene, polyethylene oxide, polyethylene terephthalate, polyvinyl isobutyl ether, polyvinyl acetate, polyvinyl chloride, polyurethane, or mixtures thereof.

4. The stabilized controlled-release pharmaceutical composition of claim 3, wherein the release-controlling polymer is present in a concentration from 25% w/w to 35% w/w.

5. The stabilized controlled-release pharmaceutical composition of claim 3, wherein the release-controlling polymer may be added intragranularly and/or extragranularly.

6. The stabilized controlled-release pharmaceutical composition of claim 3 comprising two or more release-controlling polymers having different viscosities.

7. The stabilized controlled-release pharmaceutical composition of claim 6, wherein the release-controlling polymer is a combination of hydroxypropyl methylcellulose having viscosity ranging from 100 cps to 750 cps, and another grade of hydroxypropyl methylcellulose having viscosity ranging from 1000 cps to 5000 cps.

8. The stabilized controlled-release pharmaceutical composition of claim 1, further comprising pharmaceutically acceptable excipients selected from the group consisting of diluents, binders, lubricants, or mixtures thereof.

9. The stabilized controlled-release pharmaceutical composition of claim 8, wherein the pharmaceutically acceptable excipients are free from colloidal silicon dioxide.

10. The process for the preparation of the stabilized controlled-release pharmaceutical composition of claim 1, wherein the process steps comprise:
a) blending gliclazide along with other pharmaceutically acceptable excipients;
b) mixing sodium citrate with the blend of step a);
c) optionally granulating the blend of step b); and
d) lubricating the blend of step b) or the granules of step c), and compressing into suitable size tablets or filling into capsules.

11. The process for the preparation of the stabilized controlled-release pharmaceutical composition of claim 1, wherein the process steps comprise:
a) sifting gliclazide, diluent, optionally a part of the binder, and optionally a release-controlling polymer, and dry mixing to achieve a uniform blend;
b) dissolving sodium citrate and optionally the remaining part of the binder in purified water to form a solution or dispersion;
c) granulating the dry powder blend of step a) using the solution or dispersion of step b);
d) sifting the controlled-release polymer using the appropriate sieve and mixing with the dried granules of step c);
e) lubricating the blend of step d); and
f) compressing the lubricated granules of step e) into tablets of suitable size, or optionally filling into capsules.
